(19) United States
(12) Patent Application Publication
(10) Pub. No.: US 2007/0122654 A1
(43) Pub. Date: May 31, 2007

(54) Dopant material and organic electroluminescent device using said dopant material

(76) Inventors: Chun-Liang Lai, Jiayi Hsien (TW); Kuo-Wei Huang, Xinzhuang City (TW); Ching-Hung Chen, Taichung (TW)

Correspondence Address: Rabin & Berdo, P.C., Suite 500, 1101 14th Street, Washington, DC, 20005, US

(21) Appl. No.: 11/288,291

(22) Filed: Nov. 29, 2005

(51) Int. Cl. H01L 51/54; C09K 11/06; C07C 211/00

(52) U.S. Cl. 428/690; 428/917; 313/504; 564/434; 564/427; 252/301.16; 257/102; 257/103; 257/E51.051; 257/E51.043; 257/E51.049

(57) ABSTRACT

The present invention relates to an organic electroluminescent device consisting of a substrate, an anode, a hole-injecting layer, a hole-transporting layer, at least one light-emitting layer, an electron-transporting layer, an electron-injecting layer and a cathode. Said at least one light-emitting layer contains a compound of formula (1) represented by: 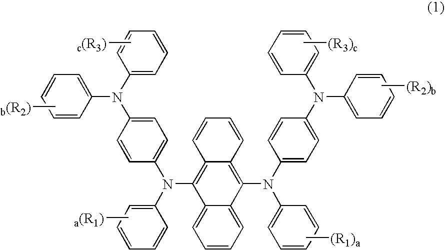
wherein R₁, R₂ and R₃ are an alkyl group containing 1 to 4 carbon atoms; a, b and c are integers ranging from 0 to 3.

### BACKGROUND OF THE INVENTION

1. Field of the Invention

The present invention relates to a dopant material and an organic electroluminescent device emitting a yellow-orange light with high luminous efficiency.

2. Related Prior Art

An organic electroluminescent device is composed of an anode, layers of organic materials and a cathode. The organic electroluminescent device has many excellent properties such as a simple structure, low thickness, wide field of view, quick response, etc. Organic electroluminescent devices are widely applied to MP3 players and sub-panels for cellular phones.

Full-color displays have been further developed by using such organic electroluminescent devices. Kodak in the U.S.; Pioneer and Hitachi in Japan; Samsung and LG in Korea; and AU Optronics, CM Optoelectronics and Ritdisplay Corporation in Taiwan keep proposing new, successfully developed full-color displays.

Implementation of a full-color display depends on the design of the light-emitting layers in the device. One type of design has light-emitting layers individually emitting red, green and blue light. Another has two light-emitting layers respectively emitting dark blue and yellow light or respectively light blue and orange light. Light emitted from such two light-emitting layers are encountered and then turn into white light. Subsequently, the white light is subjected to a color filter to achieve a full-color state. The latter can be easily made and produced in industrial quantities. The white light can also be used for illumination even though it is not subjected to the color filter for full-color applications.

The light-emitting layer of the device is composed of a host material and a dopant material of high luminous efficiency. When voltage is applied to the organic electroluminescent device, electronic holes combine with electrons in the light-emitting layer so that the host material is excited and generates photons. Subsequently, energy is transmitted from the host material to the dopant and leads the dopant to an excited state. When the dopant returns to the ground state, the energy is released in the form of light. In other words, the luminous efficiency of the device and the colors of light are influenced by the dopant in the light-emitting layer. In such a manner of using the combination of a host material and a dopant, the energy can be efficiently used and will not be transformed into heat. The luminous efficiency of such device is superior to that of using a single material.

Suitable organic materials emitting yellow light are Rubrene and its derivatives (U.S. Pat. Nos. 6,387,547 and 6,399,223, JP 2002-097465, Appl. Phys. Lett., 85, 19, 4304) and pyran derivatives (Chem. Mater. 2001, 13, 456). However, the luminous efficiency of these materials is not high enough (<10 cd/A), and cost associated with their preparation is high. Therefore, these materials are not practical and not desired.

### SUMMARY OF THE INVENTION

An objective of the present invention is to provide a compound having easy-to-prepare properties, high thermal resistance and high luminous efficiency to improve the luminous efficiency of the yellow-light organic electroluminescent device. This compound is represented by formula (1):

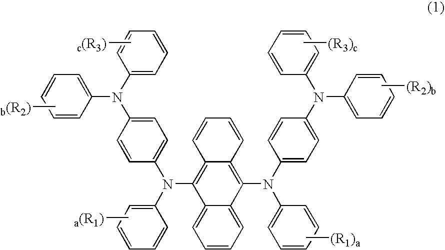

wherein R₁, R₂ and R₃ are an alkyl group containing 1 to 4 carbon atoms; a, b and c are integers ranging from 0 to 3.

The present invention also provides an organic electroluminescent device containing the compound of formula (1). Said organic electroluminescent device has at least one light-emitting layer doped with the compound of formula (1).

Preferably, in the compound of formula (1), a, b and c are integers of 0 or 1; R₁, R₂ and R₃ are methyl, ethyl, tert-butyl group, etc. Methyl is more preferable because methyl can increase the solubility of the compound and will not cause the molecular weight of the compound to be excessively high.

The compound of formula (1) can be but is not limited to D1 to D4 represented by following formulae:

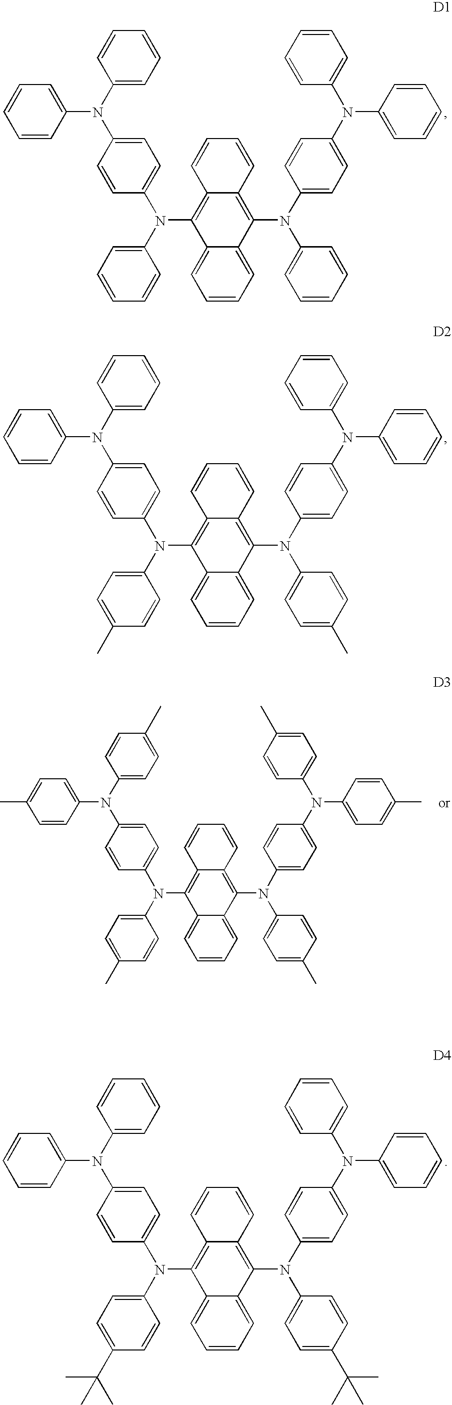

The compound of formula (1) can be prepared by many processes. For example, 9,10-dibormoanthracene and aniline derivatives can be coupled to obtain an intermediate by being catalyzed with palladium. Subsequently, the intermediate and 4-halogen substituted triphenylamine are coupled to obtain a compound of formula (1). The reaction is shown as follows:

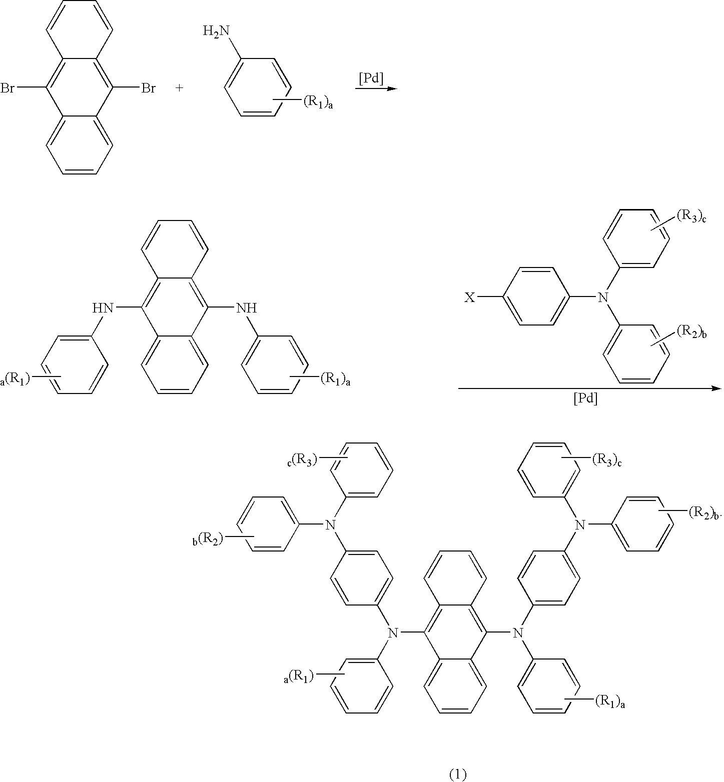

The compound of formula (1) can be purified by column chromatography, recrystallization or sublimation. The purity of said compound can be above 99%. Sublimation is preferable for purification of the compound because it has merits of (1) effectively removing mineral salts; (2) increasing the particle compactness of the product and (3) ensuring that the product is totally dry to reduce any factors that deteriorate the organic electroluminescent device.

The structure of the organic electroluminescent device in accordance with the present invention may consist of (1) an anode, a hole-injecting layer, a hole-transporting layer, a light-emitting layer, an electron-transporting layer, an electron-injecting layer and a cathode; or (2) an anode, a hole-transporting layer, a light-emitting layer, an electron-transporting layer and a cathode. The first structure (1) is preferable for the organic electroluminescent device. Generally, transparent materials, such as glass, are employed as a substrate for the manufacture of an organic electroluminescent device. The organic materials comprising the organic electroluminescent device are heated in a vacuum (<10⁻³ torr) to 200-600° C. to be directly vaporized and coated on the substrate to form a film having a thickness that may be controlled by a quartz vibrator.

The anode is generally made of a metal, an alloy or a conductive material such as ITO (indium tin oxide) or gold and has a work function, a resistance and a thickness. The work function is higher than 4 eV,. Preferably, the resistance of the anode is lower than 100 Ω/□, and its thickness is in the range of 50˜200 nm.

The cathode is generally made of a metal, an alloy or a conductive material such as Al, Li, Mg, Ag, Al—Li alloy, Mg—Ag alloy, etc. and has a work function and a thickness. The work function is lower than 4 eV. The thickness of the cathode is preferably in the range of 50˜200 nm.

The electron-injecting layer is mainly made of a metal or an inorganic ionic compound, such as LiF, CsF, Cs, etc. and has a thickness. The thickness of said layer is preferably less than 1 nm.

The hole-injecting layer may be made of conventional phthalocyanine dyes, such as copper phthalocyanine and zinc phthalocyanine, or triarylamine derivatives, such as m-TDATA (4,4′,4″-tris(N-3-methyl-phenyl-N-phenyl-amino)triphenylamine) and 1-TNATA(4,4′,4″-tris(N-(1-naphthyl)-N-phenyl-amino)triphenylamine), and has a thickness. The thickness of this layer is preferably in the range of 20˜80 nm.

The hole-transporting layer may be made of conventional NPB (N,N′-di(naphthalene-1-yl)-N,N′-diphenyl-benzidine), PPB (N,N′-bis(phenanthren-9-yl)-N,N′-diphenylbenzidine) or spiro-TAD (2,2′,7,7′-tetra-(diphenylamino)-9,9′-spiro-bifluorene) and has a thickness. The thickness of this layer is preferably in the range of 10˜50 nm.

The light-emitting layer of the organic electroluminescent device is composed of a host material and a dopant material of high luminous efficiency and has a thickness and a luminescent wavelength. Generally, the highest occupied molecular orbital (HOMO) of the host material is preferably lower than that of the dopant. The lowest unoccupied molecular orbital (LUMO) of the host material is preferably higher than that of the dopant. The light-emitting layer of such a combination can prevent the occurrence of Exiplex and improve the efficiency of the energy conversion.

The HOMO of the compound of formula (1) is about 5.1˜5.3 eV, and the LUMO is about 2.5˜2.8 eV. The host materials that can be used together with the compound of formula (1) include but are not limited to, for example, metal complexes, such as Alq₃ (tris(8-hydroxyquinolinato)aluminum); anthracene derivatives, such as 9,10-bis(2-naphthyl)anthracene, 2-methyl-9,10-bis(2-naphthyl)anthracene, 2-tert-butyl-9,10-bis(2-naphthyl)anthracene, 10,10-bis(biphenyl-4-yl)-9,9-dianthracene and 10,10-bis(biphenyl-2-yl)-9,9-dianthracene; diphenylvinyl derivatives (2,2-diphenylvinyl derivatives), such as 4,4′-bis(2,2-diphenylvinyl)-1,1′-biphenyl (DPVBi) and 6,6′-bis(2,2′-diphenylvinyl)-2,2′-binaphthalene. Anthracene derivatives and diphenylvinyl derivatives are preferable as a host material.

Generally, the compound of formula (1) is preferably in an amount of 0.5-10% by weight of the host material. The thickness of the light-emitting layer is preferably in the range of 10-50 nm. The maximum luminescent wavelength of the device is 550-600 nm.

The electron-transporting layer of the organic electroluminescent device may be formed from a metal-quinolinate complex, such as Alq₃ (tris(8-hydroxyquinolinato)aluminum), Bebq₂ (bis(10-hydroxybenzo[h]quinolinato)beryllium), Gaq₃ (tris(8-hydroxyquinolinato)gallium) and the like; a triazine derivative; or an oxadiazole derivative and has a thickness. The metal-quinolinate complex is a commonly used electron-transporting material because it has high thermal stability and can be directly vaporized in a vacuum at elevated temperatures. The thickness of this layer is preferably in the range of 10-50 nm. In a preferred embodiment, an example of the fabrication of the organic electroluminescent device in accordance with the present invention comprises the following sequential steps. An anode is formed by deposition or sputtering of anode material by vacuum evaporation on a suitable transparent substrate. Subsequently, a hold-injecting layer, a hole-transporting layer, a luminescent layer, an electron-transporting layer and an electron-injecting layer are formed sequentially by deposition by vacuum evaporation. Generally, the vacuum is preferably lower than 10⁻³ torr, and the rate of evaporation is preferably 0.01˜5.0 nm per second. Finally, a cathode is formed by deposition or sputtering by vacuum deposition to complete the organic electroluminescent device. The organic electroluminescent device is suitably packaged and can be operated in the atmosphere.

Alternatively, the device may also be fabricated in a reverse sequence. Specifically, a cathode is first formed on the substrate and then an electron-injecting layer, an electron-transporting layer, a luminescent layer, a hole-transporting layer, a hold-injecting layer and finally an anode are formed in sequence. When a direct current is applied, the device will emit light steadily and continuously.

The following examples further clarify the present invention in further detail.

### EXAMPLES

### Example 1

### Synthesis of Compound D1

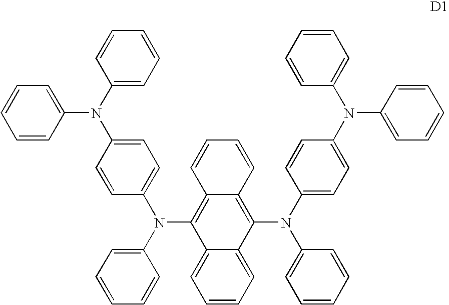

a) Synthesis of 9,10-bis(N-phenylamino)anthracene 20 g of 9,10-dibormoanthracene, 13.0 ml of aniline, 13.7 g of sodium tert-butoxide, 109 mg of tris(dibenzylideneacetone)dipalladium and 99 ml of toluene were added to a reaction vessel and heated to 50° C. in a nitrogen atmosphere. Next, 48 mg of tri-teit-butylphosphine was added to the mixture. After stirring for 2 hours, the heating was stopped, and 120 ml of methanol was added to the mixture. The resultant mixture was cooled to 25° C. The mixture was filtered to obtain filtrate, and the filtrate was dried at 150° C. to obtain 20 g of 9,10-bis(N-phenylamino)anthracene of light-yellow solid (yield: 93%, purity: 83% (HPLC, 254 nm)). The 9,10-bis(N-phenylamino)anthracene was used for the next step without further purification.

b) Synthesis of Compound D1

20 g of 9,10-bis(N-phenylamino)anthracene, 39.6 g of 4-bromo-triphenylamine, 11.7 g of sodium tert-butoxide, 204 mg of tris(dibenzylideneacetone)dipalladium and 92.5 ml of xylene were added to a reaction vessel and heated to 50° C. in a nitrogen atmosphere. Next, 48 mg of tri-tert-butylphosphine was added to the mixture. The mixture was slowly heated to 140° C. and stirred for 2 hours. Subsequently, the mixture was cooled to 60° C. and 150 mg methanol was added to the mixture. Then, the resultant mixture was further cooled to 25° C. After the mixture was filtered to obtain a filtrate and the filtrate was dried at 200° C., the solid product was sublimed to obtain 25 g of compound D1 (yield: 53%, purity:>99% (HPLC, 254 nm)) of red solid.

Tg=not detected; Tm=440° C. and UV-Vis (λmax, in THF)=477 nm.

### Example 2

### Synthesis of Compound D3

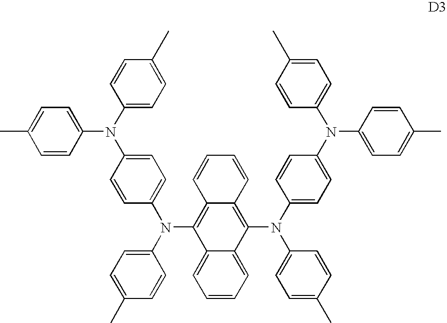

a) Synthesis of 9,10-bis(N-(p-tolyl)amino)anthracene

20 g of 9,10-dibormoanthracene, 24.4 ml of p-toluidine, 13.7 g of sodium tert-butoxide, 109 mg of tris(dibenzylideneacetone)dipalladium and 99 ml of toluene were added to a reaction vessel and heated to 50° C. in a nitrogen atmosphere. Next, 48 mg of tri-teit-butylphosphine was added to the mixture. After stirring for 2 hours, the heating was stopped, and 120 ml of methanol was added to the mixture. The resultant mixture was cooled to 25° C. The mixture was filtered to obtain filtrate, and the filtrate was dried at 120° C. to obtain 20.8 g of 9,10-bis(N-(p-tolyl)amino)anthracene of yellow solid (yield: 90%, purity: 92%). The 9,10-bis(N-(p-tolyl)amino)anthracene was used for the next step without further purification.

b) Synthesis of Compound D3

20 g of 9,10-bis(N-(p-tolyl)amino)anthracene, 39.9 g of 4-bromo-4′,4″-dimethyl-triphenylamine, 10.9 g of sodium tert-butoxide, 188 mg of tris(dibenzylideneacetone)dipalladium and 86 ml of xylene were added to a reaction vessel and heated to 50° C. in a nitrogen atmosphere. Next, 84 mg of tri-tert-butylphosphine was added to the mixture. The mixture was slowly heated to 140° C. and stirred for 2 hours. Subsequently, the mixture was cooled to 60° C., and 150 mg of methanol was added to the mixture. Then, the resultant mixture was further cooled to 25° C. Next, the mixture was filtered and added to 900 ml of N,N-dimethyl formamide. Then, the mixture was heated at 150° C. for 2 hours and cooled to 25° C. After the mixture was filtered to obtain filtrate, and the filtrate was dried at 200° C. to obtain a solid product. The solid product was sublimed to obtain 27.3 g of compound D3 (yield: 56%, purity:>99%) of red solid.

Tg=not detected and Tm>440° C.

### Example 3

An ITO glass substrate with a surface resistivity of 20 Ω/□ was placed in a vacuum vessel of a vapor deposition machine. A crucible containing 2-TNATA, a crucible containing NPB, a crucible containing 10,10′-bis(biphenyl-4-yl)-9,9′-dianthracene, a crucible containing D1, a crucible containing tris(8-hydroxylquinolinato)aluminum (Alq₃), a crucible containing aluminum and a crucible containing lithium fluoride were placed in the machine.

The pressure in the vacuum vessel on the machine was reduced to 10⁻⁶ torr. The crucible containing 2-TNATA was heated and 2-TNATA was deposited on the glass substrate by evaporation at a rate of 0.2 nm/s to form a hole-injecting layer having a thickness of 60 nm. Subsequently, a NPB film having a thickness of 20 nm was formed on the hole-injecting layer as a hole-transporting layer at a rate of 0.2 nm/s from the crucible containing NPB. Thereafter, the crucibles containing 10,10′-bis(biphenyl-4-yl)-9,9′-dianthracene and compound D1 were heated and a light-emitting layer composed of 10,10′-bis(biphenyl-4-yl)-9,9′-diantlracene incorporated with 3% of compound D1 was formed on the hole-transporting layer at a rate of 0.2 nm/s. The thickness of the light-emitting layer is 30 nm. Then, an Alq₃ film having a thickness of 25 nm was formed on the light-emitting layer as an electron-transporting layer from the crucible containing Alq₃. Subsequently, a lithium fluoride film having a thickness of 0.7 nm was formed on the light-emitting layer as an electron-injecting layer by evaporation deposition from the crucible containing lithium fluoride. Finally, an aluminum cathode film having a thickness of 150 nm was formed on the electron-injecting layer from the crucible containing aluminum.

When a voltage of 17.2 V was applied to the organic electroluminescent device, a yellow light was emitted with a light intensity of 96,600 cd/m², a luminous efficiency of 12.5 cd/A and a CIE coordinate of x=0.508, y=0.466.

### Example 4

The procedure was the same as the procedure used Example 3 except that the light-emitting layer was composed of 10,10′-bis(biphenyl-4-yl)-9,9′-dianthracene incorporated with 5% of compound D1. When a voltage of 18.1 V was applied to the organic electroluminescent device, a yellow light was emitted with a light intensity of 87,200 cd/m², a luminous efficiency of 10.9 cd/A and a CIE coordinate of x=0.523, y=0.464.

### Comparative Example of An Organic Electroluminescent Device

The procedure was the same as the procedure used Example 3 except that the light-emitting layer was composed of 10,10′-bis(biphenyl-4-yl)-9,9′-dianthracene incorporated with 3% of Rubrene. Rubrene has a chemical structure shown below.

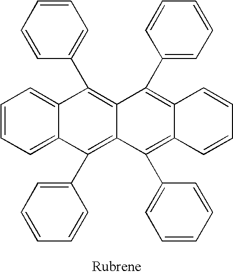

When a current of 100 mA/cm² was applied to the organic electroluminescent device, a yellow light was emitted with a light intensity of 7,910 cd/m², a luminous efficiency of 7.91 cd/A and a CIE coordinate of x=0.44, y=0.55.

The data of example 1 and the comparative example shows that the luminous efficiency of the organic electroluminescent device is improved when the compound of formula (1) was used as a dopant of the light-emitting layer of the organic electroluminescent device.

### INDUSTRIAL APPLICABILITY

When a compound of formula (1) is used to form the light-emitting layer of an organic electroluminescent device, the device obtained has an advantage of high luminous efficiency. Such an organic electroluminescent device can be advantageously used in display panels for MP3 players, digital cameras, cellular phones, etc.

What is claimed is:

1. A compound of formula (1), 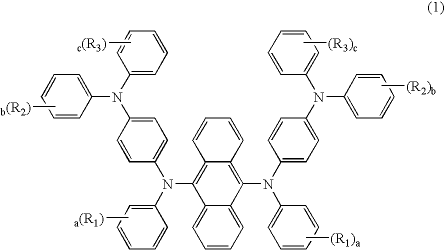
wherein R₁, R₂ and R₃ are an alkyl group containing 1 to 4 carbon atoms; a, b and c are integers ranging from 0 to 3.

2. The compound according to claim 1, wherein R₁, R₂ and R₃ are selected from a group consisting of methyl, ethyl and tert-butyl.

3. The compound according to claim 2, wherein R₁, R₂ and R₃ are methyl.

4. The compound according to claim 1, wherein a, b and c are integers of 0 or 1.

5. The compound according to claim 1 selected from the group consisting of:

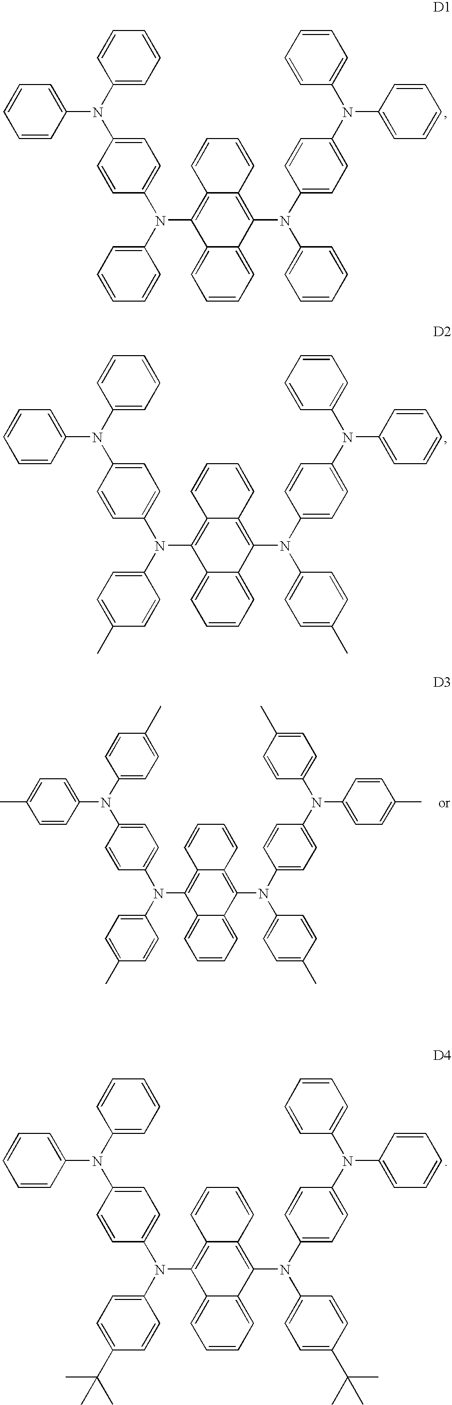

6. An organic electroluminescent device, comprising at least one light-emitting layer doped with the compound according to claim 1.

7. A light-emitting layer for an organic electroluminescent device formed from a host material and the compound according to claim 1.

8. The light-emitting layer according to claim 7, wherein said compound is 0.5-10% by weight of the host material.

9. The light-emitting layer according to claim 7, wherein said host material is an aluminum complex, anthracene derivatives or diphenylvinyl derivatives.

10. The light-emitting layer according to claim 9, wherein said host material is tris(8-hydroxyquinolinato)aluminum (Alq₃).

11. The light-emitting layer according to claim 9, wherein said host material is selected from a group consisting of 9,10-bis(2-naphthyl)anthracene, 2-methyl-9,10-bis(2-naphthyl)anthracene, 2-tert-butyl-9,10-bis(2-naphthyl)anthracene, 10,10-bis(biphenyl-4-yl)-9,9-dianthracene and 10,10-bis(biphenyl-2-yl)-9,9-dianthracene.

12. The light-emitting layer according to claim 9, wherein said host material is selected from a group consisting of 4,4′-bis(2,2-diphenylvinyl)-1,1′-biphenyl (DPVBi) and 6,6′-bis (2,2′-diphenylvinyl)-2,2′-binaphthalene.
